(19) United States
(12) Patent Application Publication
(10) Pub. No.: US 2002/0120001 A1
(43) Pub. Date: Aug. 29, 2002

(54) Compositions containing tryptamines, cartenoids and tocotrienols and having synergistic antioxidant effect

(75) Inventors: John G. Babish, Brooktondale, NY (US); Terrence Howell, Freeville, NY (US)

Correspondence Address: M. Wayne Western, THORPE NORTH & WESTERN, L.L.P., P.O. Box 1219, Sandy, UT, 84091-1219, US

(73) Assignee: Ashni Naturaceuticals, Inc.

(21) Appl. No.: 09/976,670

(22) Filed: Oct. 11, 2001

Related U.S. Application Data
(60) Provisional application No. 60/240,013, filed on Oct. 13, 2000.

(51) Int. Cl. A61K 38/05; A61K 31/70; A61K 31/404; A61K 31/355; A61K 31/198; A61K 31/015

(52) U.S. Cl. 514/415; 514/458; 514/763; 514/18; 514/23; 514/53; 514/561

(57) ABSTRACT

A novel formulation is provided that serves to synergistically inhibit the generation of free radicals and oxidative stress in warm blooded animals. The formulation comprises an effective amount of a first component of a tryptamine species or derivatives thereof, and, as a second component, at least one member selected from the group consisting of a carotenoid species, a tocotrienol species and derivatives thereof, and provides for synergistic anti-oxidant activity.

### CROSS REFERENCE TO RELATED APPLICATIONS

This application claims benefit of U.S. Provisional Application Ser. No. 60/240,013 filed Oct. 13, 2000.

### FIELD OF THE INVENTION

The present invention relates generally to a composition exhibiting synergistic antioxidant activity. More particularly, the composition comprises, as a first component, tryptamine or derivatives thereof, and, as a second component at least one member selected from the group consisting of a carotenoid species, a tocotrienol species and derivatives thereof. The composition exhibits synergistic antioxidant activity.

### BACKGROUND OF THE INVENTION

Oxygen is essential for aerobic life, but is also a precursor to the formation of harmful reactive oxygen species (ROS). Oxidative stress refers to the cytotoxic consequences of a mismatch between the production of free radicals and the ability of the cell to defend against them. Oxidative stress can thus occur when the formation of ROS increases, scavenging of ROS or repair of oxy-modified macromolecules decreases, or both. ROS may be oxygen-centered radicals possessing unpaired electrons, such as superoxide and hydroxyl radicals, or covalent molecules, such as hydrogen peroxide.

Superoxide and hydrogen peroxide are relatively nonreactive toward biological molecules. Hydroxyl radicals, on the other hand, are highly reactive. Under physiological conditions, superoxide is converted to hydrogen peroxide by the enzyme superoxide dismutase (SOD) or by interaction with transition metals. Hydrogen peroxide is in turn reduced to water by glutathione peroxidase or converted to oxygen and water by catalase. Thus, the hydroxyl radical represents the greatest threat to cell viability.

ROS, especially hydroxyl radicals, can produce functional alterations in lipids, proteins, and nucleic acids. The incorporation of molecular oxygen into polyunsaturated fatty acids initiates a chain reaction in which ROS, including hydroxyl radicals, hydrogen peroxide, and peroxyl and alkoxyl radicals are formed. Oxidative lipid damage, termed lipid peroxidation, results in a progressive loss of membrane fluidity, reduces membrane potential, and increases permeability to ions such as calcium. ROS can damage proteins and change amino groups on amino acids resulting in the inactivation of the proteins. DNA and RNA are also targets of ROS.

Hydroxyl radicals modify ribose phosphates, pyrimidine nucleotides and nucleosides and react with the sugar phosphate backbone of DNA causing breaks in the DNA strand.

Because ROS and the associated oxidative stress can produce fundamental cellular damage, primary or secondary oxidative insults have been implicated in many diseases. Table 1 below provides a list of physiological insults in which oxidative stress and ROS are believed to play a significant role and are therefore appropriate targets for normalization, prevention or treatment by antioxidants.

TABLE 1

| Physiological Insults Generating | |
|---|---|
| Oxidative Stress | Affected Tissues or Systems |
| Addison's Disease | Adrenal |
| Aging | Skin and other systems |
| Allergies | Inflammatory cells |
| Alzheimer Disease | Nerve cells |
| Angioplasty | Arterial epithelial cells |
| Arthritis | Inflammatory cells |
| Asthma | Immune cells |
| Atherosclerosis | Vessel wall |
| Cigarette Smoking | Lung, mouth, throat and |
| | blood vessels |
| Colon Cancer | Intestine |
| Chacaxia | Muscular and Nervous |
| Crohn's Disease | Intestine |
| Cystic Fibrosis | Lungs |
| Diabetes (type I and type II) | Pancreas and various systems |
| Eczema | Skin/Inflammatory cells |
| Exercise | Muscle |
| Graves' Disease | Thyroid |
| Guillain-Barre Syndrome | Nerve cells |
| Head Injury | Brain |
| Hemodialysis | Kidney |
| Hepatitis | Liver |
| HIV -1 Infection | Muscular and Immune systems |
| Hypercholesterolemia | Arterial vessels |
| Hyperlipidemia | Liver and Arterial vessels |
| Hyperthyroidism | Thyroid |
| Inflammation | Immune cells |
| Inflammatory Bowel Disease | Intestine |
| Leukemia | Immune cells |
| Lymphomas | Immune cells |
| Multiple Sclerosis | Nerve cells |
| Myasthenia Gravis | Neuromuscular junction |
| Nuclear Factor kappaB Activation | Immune cells |
| Neurodegeneration | Central nervous system |
| Physical Exertion | Muscular and Immune systems |
| Psoriasis | Skin |
| Primary Biliary Cirrhosis | Liver |
| Reperfusion Injury | Head and heart |
| Rheumatoid Arthritis | Joint lining |
| Solid Tumors | Various |
| Systemic Lupus Erythematosis | Multiple tissues |
| Tumor Necrosis Factor-alpha | Various Systems |
| Expression | |
| Uveitis | Eye |
| Weight loss | Muscle, fat cells |

Numerous epidemiological investigations have suggested that consumption of antioxidants in the form of fresh fruits and vegetables provides protection from cancer, cardiovascular disease, autoimmune disease and neurodegeneration. Furthermore, in vitro studies support the palliative effects of single, purified antioxidant treatment in a variety of model systems. However, the recently reported β-carotene and lung cancer intervention trial suggested that supraphysioloical supplementation of a single antioxidant may not only be ineffective, but contraindicaed. Therefore, it has become apparent that highly effective, combinations of antioxidants representing distinct classes of compounds are needed to increase the likelihood of clinical success.

Over three centuries ago, the French philosopher Rene Descartes described the pineal gland as “the seat of the soul’. However, it was not until the late 1950s that the chemical identity and biosynthesis of melatonin [FIG. 1A], the principal hormone secreted by the pineal body, were revealed. Melatonin, named from the Greek melanos, meaning black, and tonos, meaning color, is a biogenic amine with structural similarities to serotonin. The mechanisms mediating the synthesis of melatonin are regulated transcriptionally by the photoperiodic environment. Once synthesized, the neurohormone is a biologic modulator of mood, sleep, sexual behavior, reproductive alterations, immunologic function, and circadian rhythms. Moreover, melatonin exerts its regulatory roles through high-affinity, pertussis toxin-sensitive G-protein (or guanine nucleotide binding protein) coupled receptors that reside primarily in the eye, kidney, gastrointestinal tract, blood vessels, and brain. More recent evidence also indicates a role for melatonin in oxidative stress and oxidative stress-related diseases, probably related to its efficient free radical scavenger (or antioxidant) activity.

The potential clinical benefits of use of melatonin as an antioxidant are remarkable, suggesting that it may be of use in the treatment of many pathophysiological disease states including various cancers, hypertension, pulmonary diseases, and a variety of neurodegenerative diseases such as Alzheimer's disease.

During the last two years, a large body of evidence has accumulated concerning melatonin's role in defending against toxic free radicals. In recent in vitro studies, melatonin was shown to be a very efficient neutralizer of —OH; indeed, in the system used to test its free radical scavenging ability it was found to be significantly more effective than the well known antioxidant, glutathione (GSH), in doing so. Likewise, melatonin has been shown to stimulate glutathione peroxidase (GSH-Px) activity in neural tissue. GSH-PX metabolizes reduced glutathione to its oxidized form and in doing so it converts H₂O₂ to H₂O, thereby reducing generation of the —OH by eliminating its precursor. More recent studies have shown that melatonin is also a more efficient scavenger of the peroxyl radical than is vitamin E. The peroxyl radical is generated during lipid peroxidation and propagates the chain reaction that leads to massive lipid destruction in cell membranes. In vivo studies have demonstrated that melatonin is remarkably potent in protecting against free radical damage induced by a variety of means. Thus, DNA damage resulting from either the exposure of animals to a chemical carcinogen or to ionizing radiation is markedly reduced when melatonin is co-administered. Likewise, the induction of cataracts, generally accepted as being a consequence of free radical attack on lenticular macromolecules, in newborn rats injected with a GSH-depleting drug are prevented when the animals are given daily melatonin injections. Also, paraquat-induced lipid peroxidation in the lungs of rats is overcome when they also receive melatonin during the exposure period. Paraquat is a highly toxic herbicide that inflicts at least part of its damage by generating free radicals.

Various relationships between melatonin and other biological processes have been studied, such as sleep, circadian rhythm, surgical stress and anaesthesia. See U.S. Pat. Nos. 6,093,409; 5,837,224 and 6,048,846. Age-related melatonin studies and melatonin during depression and other psychiatric disorders have been reviewed. Some studies have been performed to use melatonin as a medication for sleep disturbance in depression, for jet-lag and as a skin protector from ultraviolet light

Neurotransmitters such as dopamine, nonadrenaline, adrenalin and trptamine derivatives, serotonin and melatonin are generally not considered biologically significant with respect to their antioxidant properties. However, Yen and Hsieh report that tryptamine was equal to alpha-tocopherol in its ability to scavenge the superoxide amino and hydroxyl radical[(1991) Antioxidant effects of dopamine and related compounds, Biosci Biotechnol Biochem 61(100:1646-1649]. In general, the antioxidant efficacy of these amines seems to be correlated with the number of hydroxyl groups and their position on the aromatic right. In light of finding that only 10 to 20% of the antioxidant activity of neuronal cells is due to enzymatic activity, neurotransmitters may serve an important antioxidant function in the brain.

Melatonin, which is a species in tryptamine genus, has been shown to be highly effective in reducing oxidative damage in the central nervous system; this efficacy derives from its ability to directly scavenge a number of free radicals and to function as an indirect antioxidant. One additional advantage melatonin has in reducing oxidative damage in the central nervous system is the ease with which it crosses the blood-brain barrier. This combination of actions makes melatonin a highly effective pharmacological agent against free radical damage. However, it would be a distinct clinical advantage to increase the antioxidant potency of melatonin through the discovery of synergistic combinations of melatonin and compounds of different chemical classes.

As a group, carotenoids have been a focus of study with respect to decreasing oxidative stress as well as cancer prevention and intervention. Carotenoids (FIG. 2[A]) are a family of over 700 natural, lipid-soluble pigments that are only produced by phytoplankton, algae, plants and a limited number of fungi and bacteria. The carotenoids are responsible for the wide variety of colors they provide in nature, most conspicuously in the yellow and red colors of fruits and leaves. In plants and algae, carotenoids along with chlorophyll and other light-harvesting pigments are vital participants in the photosynthetic process.

Biologically, carotenoids are distinguished by their capacity to interact with singlet oxygen and free radicals. Among the carotenoids, a growing body of scientific literature describes astaxanthin as one of the best antioxidants. Due to its unique molecular structure among carotenoids (a carbonyl and hydroxyl group on each of the terminal aromatic rings), astaxanthin has both a potent quenching effect against singlet state oxygen and a powerful scavenging ability for free radicals. Among the carotenoids, astaxanthin has most recently demonstrated the greatest antioxidant activity. Thus, astaxanthin serves as an extremely effective antioxidant against these reactive species.

As mentioned previously, however, experience with cancer intervention trials, as well as animal studies, have shown that supplementation with a single antioxidant may produce untoward, stimulatory effects on cancer growth. It has been concluded that supplementation with multiple antioxidant is necessary to achieve clinical effectiveness. The most rational approach for the identification of antioxidant combinations is the identification of synergy between the components of the formulation. Therefore, It would be useful to produce a potent combination of antioxidants that function synergistically to inhibit the generation of free radicals.

Tocotrienols [FIG. 3B] are a family of dietary supplements related to vitamin E and are considered powerful antioxidants. Although they can be chemically synthesized, the best natural sources for tocotrienols are the oils derived from rice bran, palm fruit, barley and wheat germ.

Comparatively, the tocotrienol structure differs from the tocopherol structure, possessing three double bonds in its side chain rather than being saturated as is the tocopherol [FIG. 3A].

Interestingly, tocotrienols have been shown to elicit powerful antioxidant, anti-cancer and cholesterol-lowering properties. See U.S. Pat. Nos. 4,603,141; 5,217,992; 5,348,974; and 5,393,776; 5,591,772; and 5,919,818 When contrasted directly, such physiological properties as anti-oxidant capacity, anti-cancer activity and cholesterol-lowering ability of tocotrienols appear to be much stronger than tocopherols. See U.S. Pat. Nos. 5,545,398 and 5,709,868. Overall, it has been concluded that the transport, tissue concentration profile, and relative biologic function of the tocopherols and tocotrienols appear somewhat disparate and possibly unrelated. Because they lack vitamin E activity [see FIG. 3C], the tocotrienols were once thought to be of lesser nutritional value than the tocopherols. From their antioxidant activity, however, they may become one of the most important nutritional compounds for the prevention and treatment of disease.

Therefore, It would be useful to provide formulations of antioxidant compounds that would function synergistically with a tryptamine species, a carotenoid species, a tocotrienol species or derivatives thereof to increase the antioxidant activity of the individual antioxidants in excess of their individual contribution. Such a synergistic combination would theoretically increase the likelihood of a positive clinical outcome.

### SUMMARY OF THE INVENTION

The present invention provides a composition having a synergistic inhibitory effect on biological oxidative processes involving free radicals or singlet oxygen. The present invention provides a composition comprising, as a first component, a tryptamine species or derivatives thereof, and, as a second component at least one member selected from the group consisting of a carotenoid species, a tocotrienol species and derivatives thereof. The composition exhibits synergistic antioxidant activity.

Preferably, the tryptamine species is a member selected from the group consisting of melatonin, tryptamine, serotonin, tryptophan, and derivatives thereof. The most preferred tryptamine species is melatonin.

Preferably, the carotenoid species is a member selected from the group consisting of astaxanthin, alpha-carotene, beta-carotene, cantaxanthin, fucoxanthin, lutein, lycopene, phytoene, preridin, and zeaxanthin. More preferably, the carotenoid species is a member selected from the group consisting of astaxanthin, alpha-carotene, beta-carotene, lutein, and lycopene. The most preferred carotenoid species is astaxanthin.

Preferably, the tocotrienol species is a member selected from the group consisting of tocotrienol, alpha-, beta-, gamma-, delta-tocotrienol, desmethyl-tocotrienol, didesmethyl-tocotrienol, and mixtures thereof. More preferably the tocotrienol species is a member selected from the group of tocotrienol, alpha-, beta-, gamma-, delta-tocotrienol and mixtures thereof. The most preferred composition of the tocotrienol species is a mixture of alpha- and beta- or a mixture of alpha-, beta-, gamma- and delta-tocotrienol. The composition functions synergistically to inhibit the generation of free radicals and oxidative stress.

The present invention further provides a composition of matter which enhances the normal functioning of the body in times of oxidative stress resulting from a chronic debilitating disease.

The present invention further provides a method of dietary supplementation and a method of treating oxidative stress or oxidative stress-based diseases in a warm blooded animal which comprises providing to the animal suffering symptoms of oxidative stress the composition of the present invention containing a second component which specifically and synergistically enhances the antioxidant activity of a tryptamine species, a carotenoid species and/or tocotrienol species and continuing to administer such a dietary supplementation of the composition until said symptoms are eliminated or reduced.

### BRIEF DESCRIPTION OF THE DRAWINGS

FIGS. 1[A] represents the general structure of melatonin (N-acetyl-5-methoxytryptamine) and 1[B] represents the general structure of the tryptamine genus, wherein R₁, R₂, R₃, R₄, and R₅ is one member selected from the group consisting of —H, —OH, —OCH₃, —O(CH₂)nCH₃ wherein n is an integer from 1 to 5, and an amino acid.

FIGS. 2[A] and [B] respectively, illustrate the general chemical structure of the carotenoid genus and astaxanthin (3, 3′-dihydroxy-β,β-carotene-4, 4′-dieto-β-carotene) as a species within that genus.

FIG. 3[A] represents the general structure of tocopherols, when R₁, R₂=—CH₃ it represents alpha-tocopherol; when R₁=—CH₃ and R₂=H it represents beta-tocopherol; when R₁=H, and R₂=—CH₃ it represent gamma-tocopherol and when R₁, R₂=H it represents delta-tocopherol; 3[B] represents the general structure of tocotrienols when R₁, R₂=—CH₃ it represents alpha-tocotrienol; when R₁=—CH₃ and R₂=H it represents beta-tocotrienol; when R₁=H and R₂=—CH₃ it represents gamma-tocotrienol and when R₁, R₂=H it represents delta-tocotrienol; 3[C] is the structure of trolox (6-hydroxy-2,5,7,8-tetramethylchroman-2-caroxylic acid, the minimum structure exhibiting vitamin E activity.

### DETAILED DESCRIPTION OF THE INVENTION

Before the present composition and methods of making and using thereof are disclosed and described, it is to be understood that this invention is not limited to the particular configurations, as process steps, and materials may vary somewhat. It is also intended to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise.

The present invention provides a composition having a synergistic antioxidant activity. More particularly, the composition comprises, as a first component, a tryptamine species or derivatives thereof, and, as a second component at least one member selected from the group consisting of a carotenoid species, a tocotrienol species and derivatives thereof.. Preferably, the molar ratio of the active first component, i.e. a tryptamine species, to a second component, i.e. at least one member selected from the group consisting of a carotenoid species, a tocotrienol species and derivatives thereof is within a range of 50:1 to 1:400. The composition provided by the present invention can be formulated as a dietary supplement or therapeutic composition. The composition functions synergistically to inhibit biological oxidation involving free radicals or singlet oxygen. Such combinations are useful as dietary supplements or therapeutics for the physiological insults listed in Table 1.

As used herein, the term “dietary supplement” refers to compositions consumed to affect structural or functional changes in physiology. The term “therapeutic composition” refers to any compounds administered to treat or prevent a disease.

As used herein, the term “antioxidant activity” refers to an inhibitory effect on biological oxidative processes involving free radicals or singlet oxygen.

As used herein, tryptamine species, carotenoid species, tocotrienol species and derivatives thereof are meant to include naturally occurring or synthetic derivatives of species within the scope of the respective genera. Natural derivatives may be obtained from common microbiological or plant sources and may exist as conjugates.

“Conjugates” of tryptamine species, carotenoid species, tocotrienol species and derivatives thereof means melatonin, carotenoid species, tocotrienol species and derivatives thereof covalently bound or conjugated to a member selected from the group consisting of mono- or di-saccharides, amino acids, fatty acids, sulfates, succinate, acetate and glutathione. Preferably, the fatty acid is a C₆ to C₂₂ fatty acid. Preferably, the mono- or di-saccharide is a member selected from the group consisting of glucose, mannose, ribose, galactose, rhamnose, arabinose, maltose and fructose.

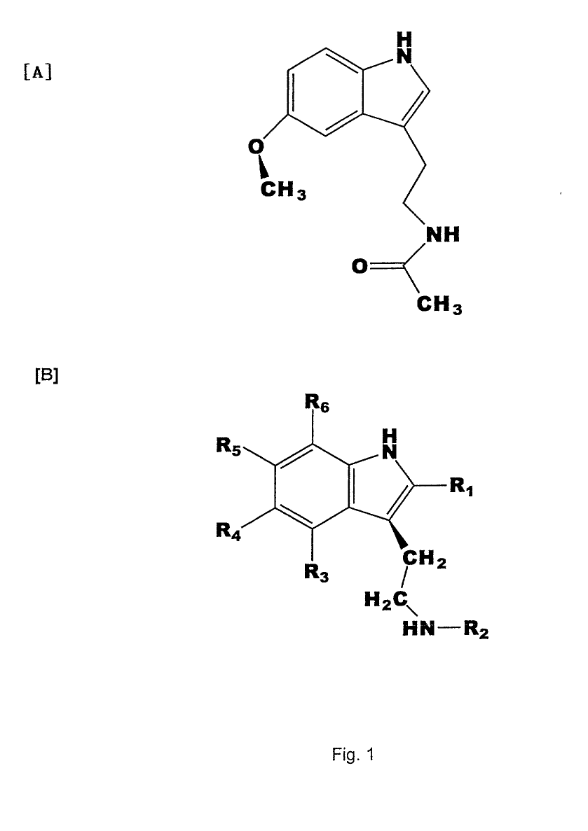

The trptamine specie and derivatives thereof are compounds represented by the general structure shown in FIG. 1[B], wherein R₁, R₂, R₃, R₄, and R₅, is one member selected from the group consisting of —H, —OH, —OCH₃, —O(CH₂)nCH₃ wherein n is an integer from 1 to 5, and an amino acid. Preferably, the tryptamine species is a member selected from the group consisting of melatonin, tryptamine, serotonin, tryptophan, and derivatives thereof. The most preferred tryptamine species is melatonin.

Preferably, the carotenoid species is a member selected from the group consisting of astaxanthin, beta-carotene, lutein, lycopene, zeaxanthin, and cantaxanthin. More preferably, the carotenoid species is a member selected from the group consisting of astaxanthin, beta-carotene, lutein, and lycopene. The most preferred carotenoid species is astaxanthin.

The tocotrienols of the present invention includes, but are not limited to, both natural tocotrienol, alpha-, beta-, gamma-, delta-tocotrienol, desmethyl-tocotrienol, and didesmethyl-tocotrienol as well as synthetic derivatives or conjugates, and mixtures thereof. Preferably, the tocotrienol species is a member selected from the group consisting of tocotrienol, alpha-, beta-, gamma-, delta-tocotrienol, desmethyl-tocotrienol, didesmethyl-tocotrienol, and mixtures thereof. More preferably the tocotrienol species is a member selected from the group of tocotrienol, alpha-, beta-, gamma-, delta-tocotrienol and mixtures thereof. The most preferred composition of the tocotrienol species is a mixture of alpha- and beta- or a mixture of alpha-, beta-, gamma-and delta-tocotrienol.

Therefore, one preferred embodiment of the present invention is a composition comprising a combination of an effective amount of a member selected from the group consisting of melatonin, tryptamine, serotonin, tryptophan, and derivatives thereof as a first component, and, as a second component, at least one member selected from the group consisting of astaxanthin, beta-carotene, lutein, lycopene, zeaxanthin, cantaxanthin, tocotrienol, alpha-, beta-, gamma-, or delta-tocotrienol and derivatives thereof. The resulting formulation of these combinations exhibits synergistic antioxidant activity.

Preferably, the tryptamine or melatonin (FIG. 1[B] and [A], respectively) employed in the present invention is a pharmaceutical grade preparation such as can be obtained commercially, for example, from DNP International Co. (Terre Haute, Ind. 47803). The pharmaceutical grade extract must pass extensive safety and efficacy procedures. Pharmaceutical grade melatonin is standardized to have a greater than 90 weight percent . As employed in the practice of the invention, the melatonin has a purity of 50 to 99 percent by weight and is synthesized using standard techniques known in chemical synthesis. Alternatively, it may be produced by fermentation, using microorganisms genetically altered to produce melatonin.

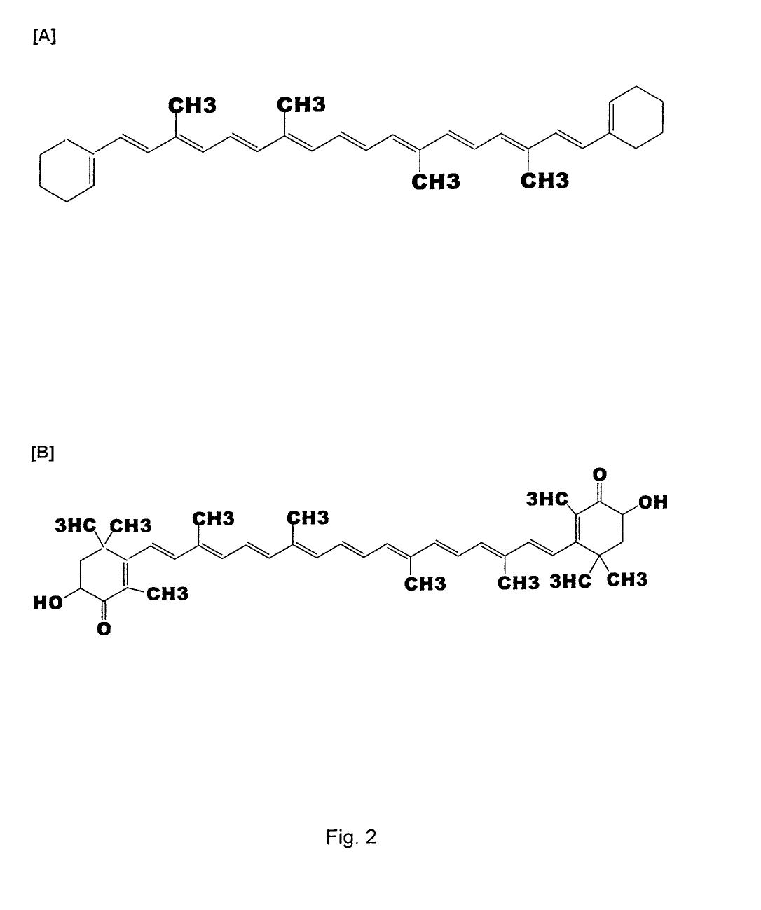

Preferably, the carotenoid or astaxanthin (FIG. 2[A] and [B], respectively) employed in the present invention is a pharmaceutical grade preparation such as can be obtained commercially, for example, from H. Reisman Corporation, Orange. N.J. The pharmaceutical grade extract must pass extensive safety and efficacy procedures. Pharmaceutical grade astaxanthin is standardized to have a greater than one weight percent of astaxanthin of total carotenoidsand can be readily obtained from the green algae Haematococcus pluvialis. As employed in the practice of the invention, the astaxanthin extract has an astaxanthin content of about 1.0 to 95 percent by weight of total carotenoids. Preferably, the minimum astaxanthin content is about 2 percent by weight. Alternatively, the astaxanthin may be synthesized using standard techniques known in chemical synthesis.

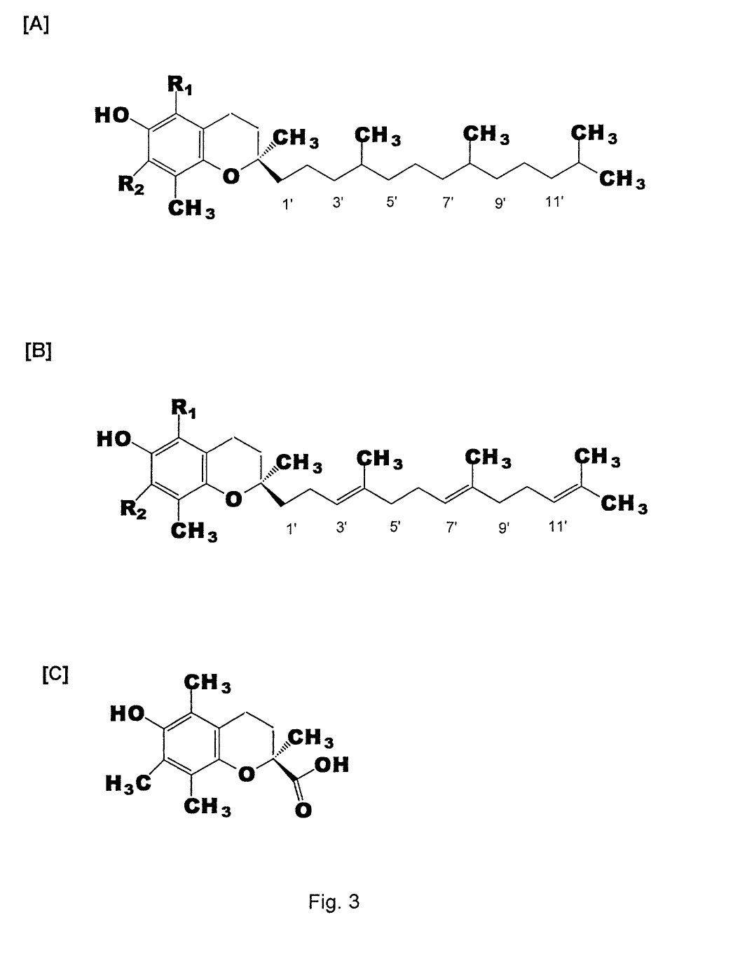

The preferred tocotrienol employed (FIG. 3[B]) is a pharmaceutical grade preparation that can be obtained from Eastman Kodak, Rochester, N.Y. In general, tocotrienol, alpha-, beta-, gamma- and delta-tocotrienol are obtained in the form of standardized mixtures of the oil derived from rice bran, palm fruit, barley and wheat germ. Pharmaceutical grade tocotrienols contain at 10 least 7 percent by weight of tocotrienols. As employed in the practice of this invention the oily tocotrienol extracts contain a minimum tocotrienol content of 1 to 50 percent by weight

The botanical sources for the tocotrienol species includes, but is not limited to, wheat germ, barley, palm fruit, rice bran, sunflower seeds, vegetable oils, brewer's grains, oats, and African violets. The preferred botanical source for tocotrienol, alpha-, beta-, gamma- and delta-tocotrienol is the lipid fraction selected from the group consisting of wheat germ, barley, palm fruit and rice bran. The most preferred botanical source for tocotrienol, alpha-, beta-, gamma- and delta-tocotrienol is the lipid fraction selected from the group consisting of palm fruit and rice bran.

Without limiting the invention, the action of the second component of the composition is thought to provide a dual, synergistic antioxidant effect with the first component. The second compound can also provide hepatoprotection, antitumor promotion, antihyperlipidemia, and antihyperglycemia.

A daily dose (mg/day) of the present dietary supplement would be formulated to deliver: 0.1 to 50 mg of a tryptamine species or derivatives thereof, 0.1 to 50 mg of a carotenoid species, and/or 0.5 to 2500 mg of a tocotrienol species or derivative thereof.

Preferably, the daily dose (mg/day) of the present dietary supplement would be formulated to deliver: 0.1 to 30 mg a tryptamine species or derivatives thereof, 3 to 15 mg of the carotenoid species or derivatives thereof, and/or 30 to 600 mg of the tocotrienol species or derivative thereof.

The composition of the present invention for topical application would contain 0.001 to 10 wt%, preferably 0.05 to 1 wt% of a tryptamine species or derivatives thereof, 0.001 to 10 wt%, preferably 0.05 to 2 wt%, of a carotenoid species or derivatives thereof, and/or, 0.001 to 10 wt%, preferably 0.05 to 2 wt% of tocotrienol species or derivatives thereof.

The preferred composition of the present invention would produce serum or tissue concentrations in the following range: 0.001 to 5 μM a tryptamine species or derivatives thereof, 0.01 to 50 μM of a carotenoid species or derivatives thereof, and/or 0.001 to 5500 μM of tocotrienol species or derivatives thereof.

In addition to the combination of active ingredients selected from the group consisting of a tryptamine species or derivatives thereof, a carotenoid species or derivatives thereof, and a tocotrienol species or derivatives thereof, the present composition for dietary application may include various additives such as other natural components of intermediary metabolism, vitamins and minerals, as well as inert ingredients such as talc and magnesium stearate that are standard excipients in the manufacture of tablets and capsules.

As used herein, “pharmaceutically acceptable carrier” includes any and all solvents, dispersion media, coatings, isotonic and absorption delaying agents, sweeteners and the like. These pharmaceutically acceptable carriers may be prepared from a wide range of materials including, but not limited to, diluents, binders and adhesives, lubricants, disintegrants, coloring agents, bulking agents, flavoring agents, sweetening agents and miscellaneous materials such as buffers and absorbents that may be needed in order to prepare a particular therapeutic composition. The use of such media and agents for pharmaceutically active substances is well known in the art. Except insofar as any conventional media or agent is incompatible with the active ingredients, its use in the present composition is contemplated. In one embodiment, talc and magnesium stearate are included in the present formulation. When these components are added they are preferably the Astac Brand 400 USP talc powder and the veritable grade of magnesium stearate. Other ingredients known to affect the manufacture of this composition as a dietary bar or functional food can include flavorings, sugars, amino-sugars, proteins and/or modified starches, as well as fats and oils.

The dietary supplements, lotions or therapeutic compositions of the present invention can be formulated in any manner known by one of skill in the art. In one embodiment, the composition is formulated into a capsule or tablet using techniques available to one of skill in the art. In capsule or tablet form, the recommended daily dose for an adult human or animal would preferably be contained in one to six capsules or tablets. However, the present compositions may also be formulated in other convenient forms such as, an injectable solution or suspension, a spray solution or suspension, a lotion, gum, lozenge, food or snack item. Food, snack, gum or lozenge items can include any ingestible ingredient, including sweeteners, flavorings, oils, starches, proteins, fruits or fruit extracts, vegetables or vegetable extracts, grains, animal fats or proteins. Thus, the present composition can be formulated into cereals, snack items such as chips, bars, gumdrops, chewable candies or slowly dissolving lozenges.

The present invention contemplates treatment of all types of oxidative stress-based diseases, both acute and chronic. The present formulation reduces the symptoms of oxidative stress and thereby promotes healing of, or prevents further damage to, the affected tissue. A pharmaceutically acceptable carrier may also be used in the present compositions and formulations.

According to the present invention, the warm blooded animal may be a member selected from the group consisting of humans, non-human primates, such as dogs, cats, birds, horses, ruminants or other animals. The invention is directed primarily to the treatment of human beings. Administration can be by any method available to the skilled artisan, for example, by oral, topical, transdermal, transmucosal, or parenteral routes.

The following examples are intended to illustrate but not in anyway limit the invention.

### EXAMPLE 1

### Antioxidant Synergy Exhibited for the Combination of Melatonin and Astaxanthin

This example illustrates the antioxidant synergy between melatonin and astaxanthin. Measurement of peroxy radical scavenging activity of the individual compounds and combinations was performed essentially as described by Naguib (Analytical Biochemistry(1998) 265:290-298) with modifications to allow the assay to be performed on a microplate. The fatty acid indicator 4,4difluoro-5-(4-phenuyl-1,3-butadienyl)-4-bora-3a-4a-diaza-s-indacene-3-undecanate (BODIPY 581/591 C₁₁) was purchased from Molecular Probes (Eugene, Oreg.). As a peroxy radical generator, 2,2′-azobis-2,4-dimethyl valeronitrile (AMVN) was used and obtained from WAKO Chemicals (Richmond, Va.). All standards were of the highest purity commercially available. Additionally, the solvents were of HPLC grade.

Fluorescence measurements were performed using a Packard FluoroCount microplate fluorometer equipped with a temperature-controlled plate holder. All measurements were preformed in a 96-well polypropylene plate with stirring. The fluorescence signal of the indicator BODIPY 581/591 C₁₁, in octane: butyronitrile (9:1, v/v) at 30° C. gradually decreased upon addition of the peroxyl radical-generating system AMVN.

Reactions in octane: butyronitrile (9:1, v/v) were carried out at 30° C. All fluorescence measurements were recorded every 15 minutes for 1 hour at excitation wavelengths of 570 nm, with emission at 620 nm. The net protection (area under the curve, AUC) by the antioxidant sample was calculated using the trapezoid method. Percent inhibition was calculated based upon AUC_{(AMVN)}=0% inhibition and AUC_{(BODIPY)}=100% inhibition. The dynamic range of inhibition was defined as the AUC between 0 and 100%=AUC_{(BODIPY)}−AUC_{(AMVN}. The percent inhibition of each dose was calculated as:

[AUC_{(dose)}−AUC_{AMVN}]/[AUC_{BODIPY)}−AUC_{(AMVN)}]×100.

The final reaction mixture (200 μl) for the assay contained 0.5 μg/mL BODIPY 581/591C₁₁ and 0.26 M AMVN in octane:butyronitrile (9:1, v/v). Stock solutions of the test samples were made up in chloroform and 6 μL was added to the reaction mixture to achieve the stated concentrations. The reaction was initiated by the addition of the AMVN fluorometric readings were then recorded every 15 minutes for one hour.

Synergy between melatonin and astaxanthin was assessed using CalcuSyn (BIOSOFT, biosoft.com). This statistical package performs multiple drug dose-effect calculations using the Median Effect methods described by T-C Chou and P. Talaly (Trends Pharmacol. Sci. 4:450-454), hereby incorporated by reference.

Briefly, the program correlates the “Dose” and the “Effect” in the simplest possible form: fa/fu=(C/Cm)m, where C is the concentration or dose of the compound and Cm is the median-effective dose signifying the potency. Cm is determined from the x-intercept of the median-effect plot. The fraction affected by the concentration of the test material is fa and the fraction unaffected by the concentration is fu (fu=1−fa). The exponent m is the parameter signifying the sigmoidicity or shape of the dose-effect curve. It is estimated by the slope of the median-effect plot.

The median-effect plot is a plot of x=log(C) vs y=log(fa/fu) and is based on the logarithmic form of Chou's median-effect equation. The goodness of fit for the data to the median-effect equation is represented by the linear correlation coefficient r of the median-effect plot. Usually, the experimental data from enzyme or receptor systems have an r>0.96, from tissue culture an r>0.90 and from animal systems an r>0.85.

Synergy of test components was quantified using the combination index (CI) parameter. The CI of Chou-Talaly is based on the multiple drug-effect and is derived from enzyme kinetic models (Chou, T.-C. and Talalay, P. (1977) A simple generalized equation for the analysis of multiple inhibitions of Michaelis-Menten kinetic systems. J. Biol. Chem. 252:6438-6442). The equation determines only the additive effect rather than synergism or antagonism. However, we define synergism as a more than the expected additive effect, and antagonism as a less than the expected additive effect as proposed by Cho and Talalay in 1983 (Trends Pharmacol. Sci. (1983) 4:450-454). Using the designation of CI=1 as the additive effect, we obtain for mutually exclusive compounds that have the same mode of action or for mutually non-exclusive drugs that have totally independent modes of action the following relationships: CI<1, =1, and >1 indicate synergism, additivity and antagonism, respectively.

Both melatonin and astaxanthin were obtained from Sigma (A9335; St. Louis, Mo.).

Dose-response curves were described with each test article separately and then in a two-way combination. For the individual dose-response curves, concentrations of melatonin included 100, 500 and 1000 μM, while the concentrations of astaxanthin were 49, 98, 490, and 980 μM. Following the estimation of the component IC50 values, serial dilutions of a mixture containing concentrations of each component and equal to 4 times the IC50 value were assayed. This resulted in a series of test concentrations of the mixture containing 4, 2, 1, 0.5, and 0.25 times the IC50 concentration of each component.

Table 1.1 depicts the calculated inhibitory concentrations and combination indexes for 50, 75 and 90 percent inhibition of peroxy radical formation by the combination of melatonin and astaxanthin. When the mixture response was parsed into the melatonin and astaxanthin components, values of 128, 185 and 267 μM, respectively, were estimated for 50, 75 and 90 percent inhibition by melatonin. Similarly, the astaxanthin concentrations for the same three parameters were 145, 210 and 303 μM. The calculated combination index for each of the values indicated synergy over the estimated regions of the dose-response curve.

TABLE 1.1
Statistical results of the antioxidant effect of a combination of melatonin
and astaxanthin

| | IC50* | IC75* | IC90* |
|---|---|---|---|
| Test Material | [μM] | [μM] | [μM] |
| Melatonin alone | 284 (242-332) | | |
| Astaxanthin alone | 327 (192-559) | 185 (107-319) | 267 (153-468) |
| Mixture—Melatonin | 128 (72-229) | 210 | 303 |
| Mixture—Astaxanthin | 145 (81-260) | 0.79 | 0.72 |
| Combination Index = | 0.90 | | |

*Exhibited significant (p < 0.5) synergy with CI < 1.0; parenthetic values are 95% confidence intervals for the estimated value.

This combination of melatonin and astaxanthin effectively increased the antioxidant potency of melatonin and astaxanthin, respectively, 2.2- and 2.3-fold (p<0.05). The example shows that a 1.0:1.1 combination of melatonin and astaxanthin provided a statistically significant (p<0.05) increase in antioxidant efficacy of both melatonin and astaxanthin.

### EXAMPLE 2

### Antioxidant Synergy Exhibited for the Combination of Melatonin and a Natural Astaxanthin

### Preparation

This example illustrates the antioxidant synergy between melatonin and a mixture of natural astaxanthin derivatives when tested in the model described in Example 1. The melatonin was purchased from Sigma (St. Louis, Mo.). The astaxanthin sample used was a commercial preparation of containing 17.8 percent total carotenoids. Astaxanthin represented 2.8 percent of the carotenoid content. Remaining carotenoids were a mixture of β-carotene, alpha-carotene, lutein, lycopene and zeaxanthin. It was obtained from H. Reisman Corporation, Orange. N.J. Dose-response curves were described with each test article separately and then in a two-way combination.

For the individual dose-response curves, concentrations of pure melatonin used in the assay included 16.5, 33, 66, 132, and 263 μg/mL. Concentrations of the astaxanthin component in the astaxanthin-containing test formulation were 17.6, 35.3, 70.5, 141 and 282 μg/mL. Following the estimation of the component IC50 values, serial dilutions of a mixture containing concentrations of each component equal to 4 times the IC50 value were assayed. This resulted in a series of test concentrations of the mixture containing 4, 2, 1, 0.5, and 0.25 times the IC50 concentration of each component.

Table 2.1 depicts the calculated inhibitory concentrations and combination indexes for 50, 75 and 90 percent inhibition of peroxy radical formation by the combination of melatonin with the natural astaxanthin preparation. When the mixture response was parsed into the melatonin and astaxanthin components, values of 38, 54, and 77 μg/mL, respectively, were estimated for 50, 75 and 90 percent inhibition by melatonin. Similarly, the astaxanthin concentrations for the same three parameters were 20, 29 and 41 μg/mL. The calculated combination index for each of the values indicated strong synergy over the complete dose-response curve.

TABLE 2.1
Statistical results of the antioxidant effect of a combination of melatonin
and natural astaxanthin

| | IC50* | IC75* | IC90* |
|---|---|---|---|
| Test Material | [μg/mL] | [μg/mL] | [μg/mL] |
| Melatonin alone | 120 (105-137) | | |
| Natural astaxanthin alone | 91 (86-97) | | |
| Mixture Melatonin | 38 (19-73) | 54 | 77 |
| Mixture Natural | 20 (10-39) | 29 (15-55) | 41 (20-83) |
| astaxanthin | | | |
| Combination Index = | 0.53 | 0.42 | 0.33 |

*Exhibited significant (p < 0.5) synergy with CI < 1.0; parenthetic values are 95% confidence intervals for the estimated value.

This combination effectively increased the antioxidant potency of both melatonin and the natural astaxanthin, respectively, 3.2- and 4.6-fold. This example shows that a 1:1 combination of melatonin and natural astaxanthin provided a statistically significant (p<0.05) increase in antioxidant efficacy.

### EXAMPLE 3

### Antioxidant Synergy Exhibited for the Combination of Melatonin, β-Carotene and Lutein

This example illustrates the antioxidant synergy for the three-way combination of melatonin, β-carotene, and lutein when tested in the model described in Example 1. The experiment was performed and analyzed as described in Example 1. Melatonin, β-carotene and lutein were obtained from Sigma (St. Louis, Mo.). Dose-response curves were described with each component separately and then in a three-way combination. For the individual dose-response curves, concentrations of melatonin included 100, 500, and 1000 μM; the β-carotene concentrations were 47.5, 95, 475 and 950 μM; and the lutein concentrations were 7, 35, 70, 350, and 700 μM. Following the estimation of the component IC50 values, serial dilutions of a mixture containing of 4 times the IC50 of each of the three compounds was assayed. This resulted in series of test concentrations of the mixture containing 4, 2, 1, 0.5 and 0.25 times the IC50 concentration of each component.

Table 3.1 depicts the calculated inhibitory concentrations and combination indexes for 50, 75 and 90 percent inhibition of peroxy radical formation by the combination of melatonin, β-carotene and lutein. Values of 74, 98 and 128 μM, respectively, were estimated for melatonin responses of 50, 75 and 90 percent inhibition of free radical formation when present in the combination mixture. The calculated combination index for each of the inhibitory parameters indicated synergy (<1.0) over the entire dose-response curve.

TABLE 3.1
Statistical results of the antioxidant effect of a combination of melatonin,
β-carotene and lutein

| | IC50* | IC75* | IC90* |
|---|---|---|---|
| Test Material | [μM] | [μM] | [μM] |
| Melatonin alone | 283 (243-330) | | |
| β-Carotene alone | 366 (318-421) | | |
| Lutein alone | 61 (47-80) | | |
| Mixture Melatonin | 74 (43-128) | 98 (22-42) | 128 (32-72) |
| Mixture β-Carotene | 91 (53-157) | 120 | 157 |
| Mixture Lutein | 11 (7-19) | 15 | 19 |
| Combination Index = | 0.69 | 0.58 | 0.49 |

*Exhibited significant (p < 0.5) synergy with CI < 1.0; parenthetic values are 95% confidence intervals for the estimated value.

The combination of melatonin, β-carotene and lutein increased the antioxidant potency of melatonin, β-carotene and lutein, respectively, 3.8-, 3.0- and 5.5-fold. This example shows that a 6:8:1 combination of melatonin, β-carotene and lutein provided a statistical and biological increase in antioxidant efficacy significantly greater (p<0.05) than the individual compounds over the critical regions of the dose-response curve.

### EXAMPLE 4

### Antioxidant Synergy Exhibited for the Combination of Melatonin and Mixed Tocotrienols

This example illustrates the antioxidant synergy for the two-way combination of melatonin and mixed tocotrienols when tested in the model described in Example 1. The experiment was performed essentially as detailed in Example 1. The melatonin was obtained from Sigma (St. Louis, Mo.) and the 50 percent oil mixture of tocotrienols was from H. Reisman Corp (Lot no. B1005-1-080799; Orange, N.J.). Within the tocotrienol fraction, approximately 56% was reported as d-gamma-tocotrienol, 30% as d-alpha-tocotrienol, 13% as d-delta-tocotrienol and 1% other tocotrienols including d-beta-tocotrienol. Dose-response curves were described with each test article separately and then in a two-way combination. For the individual dose-response curves, concentrations of melatonin included 23, 116 and 232 μg melatonin/mL; the total mixed tocotrienol concentrations were 12.5, 62.5, and 125 μg tocotrienols/mL. Following the estimation of the component IC50 values, serial dilutions of a mixture containing concentrations of each component equal to 4 times the IC50 value were assayed. This resulted in a series of test concentrations of the mixture containing 4, 2, 1, 0.5, and 0.25 times the IC50 concentration of each component.

Table 4.1 depicts the calculated inhibitory concentrations and combination indexes for 50, 75 and 90 percent inhibition of peroxy radical formation by the combination of melatonin with mixed tocotrienols. When the mixture response was parsed into the melatonin and mixed tocotrienol components, values of 17, 21 and 26 μg/mL, respectively, were estimated for 50, 75 and 90 percent inhibition by melatonin. Similarly, the mixed tocotrienol concentrations for the same three parameters were 23, 28 and 34 μg/mL. The calculated combination index for each of the values indicated synergy over the relevant regions of the dose-response curve.

TABLE 4.1
Statistical results of the antioxidant effect of a combination of melatonin,
and mixed tocotrienols

| | IC50* | IC75* | IC90* |
|---|---|---|---|
| Test Material | [μg test/mL] | [μg test/mL] | [μg test/mL] |
| Melatonin alone | 53 (25-112) | | |
| Mixed Tocotrienols alone | 54 (44-65) | | |
| Mixture—Melatonin | 17 (11-27) | 21 (14-32) | 26 (17-40) |
| Mixture—Tocotrienols | 23 (32-73) | 28 | 34 |
| Combination Index = | 0.75 | 0.65 | 0.58 |

*Exhibited significant (p < 0.5) synergy with CI < 1.0; parenthetic values are 95% confidence intervals for the estimated value.

This 1:1 combination of melatonin and mixed tocotrienols increased the antioxidant potency of both melatonin and mixed tocotrienols 3.1 and 2.3-fold, respectively. This example shows that a combination of 1 part of melatonin to 1 part of mixed tocotrienols provided a statistically significant (p<0.05) increase in antioxidant efficacy of both melatonin and mixed tocotrienols.

### EXAMPLE 5

### Antioxidant Synergy Exhibited for the Combination of Melatonin, Mixed Tocotrienols and Astaxanthin

This example illustrates the antioxidant synergy for the three-way combination of melatonin, a mixture of tocotrienols and astaxanthin when tested in the model described in Example 1. The experiment was performed as described in Example 1. Both melatonin and astaxanthin were obtained from Sigma (St. Louis, Mo.). The 50 percent oil mixture of tocotrienols was from H. Reisman Corp (Lot no. B1005-1-080799; Orange, N.J.). Within the tocotrienol fraction, approximately 56% was reported as d-gamma-tocotrienol, 30% as d-alpha-tocotrienol, 13% as d-delta-tocotrienol and 1% other tocotrienols including d-beta-tocotrienol. Dose-response curves were described with each component separately and then in a three-way combination. For the individual dose-response curves, concentrations of melatonin included 23, 116, and 232 μg melatonin/mL; the mixed tocotrienols were tested at 12.5, 62.5, and 125 μg tocotrienols/mL: and the individual astaxantin concentrations were 29, 58.5 and 292 μg astaxanthin/mL. Following the estimation of the component IC50 values, serial dilutions of a mixture containing of 4 times the IC50 of each of the three compounds was assayed. This resulted in series of test concentrations of the mixture containing 4, 2, 1, 0.5 and 0.25 times the IC50 concentration of each component.

Table 5.1 depicts the calculated inhibitory concentrations and combination indexes for 50, 75 and 90 percent inhibition of peroxy radical formation by the combination of melatonin, astaxanthin mixed tocotrienols. Values of 20, 31 and 47 μg/mL, respectively, were estimated for tocotrienol responses of 50, 75 and 90 percent inhibition of free radical formation when present in the combination mixture. The calculated combination index for each of the values indicated synergy over the entire dose-response curve.

TABLE 5.1
Statistical results of the antioxidant effect of combining melatonin, mixed
tocotrienols and astaxanthin

| | IC50* | IC75* | IC90* |
|---|---|---|---|
| Test Material | [μg test/mL] | [μg test/mL] | [μg test/mL] |
| Melatonin alone | 53 (25-112) | | |
| Tocotrienols alone | 54 (44-65) | | |
| Astaxanthin alone | 148 (94-232) | | |
| Mixture—Melatonin | 16 (14-18) | 24 (21-29) | 37 (30-47) |
| Mixture—Tocotrienols | 16 (14-18) | 24 | 38 |
| Mixture—Astaxanthin | 44 (38-50) | 68 | 105 |
| Combination Index = | 0.88 | 0.92 | 0.98 |

*Exhibited significant (p < 0.5) synergy with CI < 1.0; parenthetic values are 95% confidence intervals for the estimated value.

The 1:2.8:1 combination of melatonin, mixed tocotrienols and astaxanthin increased the 30 antioxidant potency of melatonin, mixed tocotrienols and astaxanthin 3.3-, 3.4- and 3.4-fold, respectively. This example shows that a combination of melatonin, mixed tocotrienols and astaxanthin provided an increase in antioxidant efficacy significantly greater (p<0.05) than the individual compounds over the critical regions of the dose-response curve.

Thus, among the various formulations taught there has been disclosed a formulation comprising as a first component melatonin, and as a second component (a) one or more carotenoid species or derivative thereof, or (b) at least one member selected from the group consisting of tocotrienols, alpha-, beta-, gamma- or delta-tocotrienol and derivatives thereof. These combinations provide for a synergistic anti-oxidant activity. It will be readily apparent to those skilled in the art that various changes and modifications of an obvious nature may be made without departing from the spirit of the invention, and all such changes and modifications are considered to fall within the scope of the invention as defined by the appended claims. Such changes and modifications would include, but not be limited to, the incipient ingredients added to affect the capsule, tablet, lotion, food or bar manufacturing process as well as vitamins, herbs, flavorings and carriers. Other such changes or modifications would include the use of other herbs or botanical products containing the combinations of the present invention disclosed above.

We claim:

1. A composition having synergistic antioxidant activity comprising an effective amount of a first component of a tryptamine species or derivatives thereof, and, as a second component, at least one member selected from the group consisting of a carotenoid species, a tocotrienol species and derivatives thereof.

2. The composition of claim 1 wherein at least one of said second component are derived from microorganisms, plants, extracts of microorganisms or plants.

3. The composition of claim 1 wherein said first and second components are synthetic compounds.

4. The composition of claim 1 wherein at least one of said first or second components is conjugated with a compound selected from the group consisting of mono- or di-saccharides, amino acids, fatty acids, sulfates, succinate, acetate, glutathione, mono- or di-saccharides and amino acids.

5. The composition of claim 1, formulated in a pharmaceutically acceptable carrier.

6. The composition of claim 1, additionally containing one or more members selected from the group consisting of antioxidants, vitamins, minerals, proteins, fats, carbohydrates, glucosamine, chondrotin sulfate and aminosugars.

7. A composition having synergistic antioxidant activity comprising an effective amount of a first component selected from the group consisting of melatonin, tryptamine, serotonin, tryptophan and derivatives thereof, and as a second component, at least one member selected from the group consisting of astaxanthin, beta-carotene, lutein, lycopene, zeaxanthn, and cantaxanthin, tocotrienol, alpha-, beta-, gamma-, delta-tocotrienol, desmethyl-tocotrienol, didesmethyl-tocotrienol, and mixtures thereof

8. The composition of claim 7 wherein at least one of said second component are derived from microorganisms, plants, extracts of microorganisms or plants.

9. The composition of claim 7 wherein said first and second components are synthetic compounds.

10. The composition of claim 7 wherein at least one of said first or second components is conjugated with a compound selected from the group consisting of mono- or di-saccharides, amino acids, fatty acids, sulfates, succinate, acetate, glutathione, mono- or di-saccharides and amino acids.

11. The composition of claim 7, formulated in a pharmaceutically acceptable carrier.

12. The composition of claim 7, additionally containing one or more members selected from the group consisting of antioxidants, vitamins, minerals, proteins, fats, carbohydrates, glucosamine, chondrotin sulfate and aminosugars.

13. A composition having synergistic antioxidant activity comprising an effective amount of a first component of a pharmaceutical grade melatonin, and as a second component, at least one member selected from the group consisting of astaxanthin, beta-carotene, lutein, lycopene, tocotrienol, alpha-, beta-, gamma-, delta-tocotrienol and mixtures thereof.

14. The composition of claim 13 wherein at least one of said second component are derived from microorganisms, plants, extracts of microorganisms or plants.

15. The composition of claim 13 wherein said first and second components are synthetic compounds.

16. The composition of claim 13 wherein at least one of said first or second components is conjugated with a compound selected from the group consisting of mono- or di-saccharides, amino acids, fatty acids, sulfates, succinate, acetate, glutathione, mono- or di-saccharides and amino acids.

17. The composition of claim 13, formulated in a pharmaceutically acceptable carrier.

18. The composition of claim 13, additionally containing one or more members selected from the group consisting of antioxidants, vitamins, minerals, proteins, fats, carbohydrates, glucosamine, chondrotin sulfate and aminosugars.

19. A composition having synergistic antioxidant activity comprising an effective amount of a first component of a pharmaceutical grade melatonin, and as a second component, at least one member selected from the group consisting of astaxanthin, beta-carotene, lutein, lycopene, a mixture of alpha-, beta-, gamma- and delta-tocotrienol and derivatives thereof.

20. The composition of claim 19 wherein at least one of said second component are derived from microorganisms, plants, extracts of microorganisms or plants.

21. The composition of claim 19 wherein said first and second components are synthetic compounds.

22. The composition of claim 19 wherein at least one of said first or second components is conjugated with a compound selected from the group consisting of mono- or di-saccharides, amino acids, fatty acids, sulfates, succinate, acetate, glutathione, mono- or di-saccharides and amino acids.

23. The composition of claim 19, formulated in a pharmaceutically acceptable carrier.

24. The composition of claim 19, additionally containing one or more members selected from the group consisting of antioxidants, vitamins, minerals, proteins, fats, carbohydrates, glucosamine, chondrotin sulfate and aminosugars.

25. A method for normalization and therapeutic treatment of symptoms of oxidative stress in warm blooded animals comprising administering to an animal a composition comprising an effective amount of a first component of a tryptamine species or derivatives thereof, and, as a second component, at least one member selected from the group consisting of a carotenoid species, a tocotrienol species and derivatives thereof; and continuing said administration until said symptoms of oxidative stress are reduced.

26. The method of claim 25 wherein the composition is formulated in a dosage form such that said administration provides 0.1 to 50 mg/day of a tryptamine species or derivatives thereof, 0.1to 50 mg/day of a cartenoid species or derivatives thereof, and 0.5 to 2500 mg/day of a tocotrienol species or derivatives thereof.

27. The method of claim 25 wherein the composition is formulated in a dosage form such that said administration provides 0.1 to 30 mg/day of a tryptamine species or derivatives thereof, 3 to 15 mg/day of a cartenoid species or derivatives thereof, and 30 to 600 mg/day of a tocotrienol species or derivatives thereof.

28. The method of claim 25, wherein the composition is administered in an amount sufficient to maintain a serum or tissue concentration of 0.001 to 5 μM of a tryptamine species or derivatives thereof, 0.01 to 50 μM of a cartenoid species or derivatives thereof, and 0.001 to 5500 μM of a tocotrienol species or derivatives thereof.

29. The method of claim 25 wherein administration is by a means selected from the group consisting of oral, parenteral, topical, transdermal and transmucosal delivery.

30. The method of claim 29 wherein the topical application formula contains 0.001 to 10 wt% of the first component and 0.001 to 10 wt% of the second component.

31. The method of claim 25 wherein the oxidative stress symptom is associated to one or more member selected from the group consisting of cardiovascular disorders, immune system disorders, cataracts and macular degeneration, aging, decreased growth rate, lack of energy, cognitive function disorders and stomach function disorders.
